(19) United States
(12) Patent Application Publication
(10) Pub. No.: US 2010/0003255 A1
(43) Pub. Date: Jan. 7, 2010

(54) GENE EXPRESSION PATTERN PREDICTIVE FOR COLORECTAL CARCINOMAS

(76) Inventors: Roland Croner, Roettenbach (DE); Werner Hohenberger, Herzogenaurach (DE); Bertram Reingruber, Eckental (DE); Berthold Lausen, Erlangen (DE)

Correspondence Address: JENKINS, WILSON, TAYLOR & HUNT, P. A., Suite 1200 UNIVERSITY TOWER, 3100 TOWER BLVD.,, DURHAM, NC, 27707, US

(21) Appl. No.: 12/302,183

(22) PCT Filed: May 23, 2007
(86) PCT No.: PCT/EP2007/055017

§ 371 Date: May 26, 2009

(30) Foreign Application Priority Data
May 24, 2006 (DE) 10 2006 024 416.8

(51) Int. Cl. A61K 39/395; C40B 40/06; C12Q 1/68; C40B 30/00; C40B 40/10; C07K 16/00; A61P 35/00

(52) U.S. Cl. 424/139.1; 506/16; 435/6; 506/7; 506/18; 530/387.9

(57) ABSTRACT

The present invention relates to a micro-array for the prediction of a lymph node metastasis in connection with colorectal carcinoma. The invention further relates to an ex vivo method for predicting a lymph node metastasis in connection with colorectal carcinoma as well as an inhibitor or a modulator suited to treat the metastasing colorectal carcinoma.

The instant application is a national stage application of PCT/EP2007/055017, filed May 23, 2007, which itself claims the benefit of German Patent Application Serial No. 10 2006 024 416.8, filed May 24, 2006, the disclosure of each of which is incorporated herein by reference in its entirety.

The present invention relates to a micro-array for the prediction of a lymph node metastasis in connection with colorectal carcinoma. Furthermore, the invention relates to an ex vivo method for predicting a lymph node metastasis in connection with colorectal carcinoma as well as an inhibitor or modulator suited for treating the metastasizing colorectal carcinoma.

### Sequence Listing Provided Electronically

The Sequence Listing associated with the instant disclosure has been electronically submitted. The electronic sequence listing contains one file named: Substitute1406593_ST25.txt, which is 1,548 kilobytes (measured in MS-Windows) and was created on Mar. 24, 2009. The Sequence Listing is hereby incorporated by reference into the instant disclosure.

For colorectal carcinoma (CRC), the lymph node metastasis (LNM) is a significant predictive factor. For this reason, the draining lymph nodes will be removed in all tumors by means of surgery. An adjuvant chemotherapy is recommended for patients with LNM. A valid pre-operative imaging detection of LNM, which could result in an individualization of the therapy, is actually not sufficiently possible.

The colorectal carcinoma is curable depending on its stage. Hereby, the surgical resection represents the decisive therapeutical measure. Besides the broad removal of the primary tumor, the principles of surgical radicalness involve the concept of the elective en bloc lymph node dissection in nearly all colorectal carcinomas (1, 2). This procedure is based on the observation that patient populations are profiting from the removal of the respective area of the tumor-associated lymphatic flow and thus of all potentially afflicted lymph nodes. After a curative operation (R0) during stage III (UICC), the loco-regional recrudescence rates could be reduced to under 5% for the colon carcinoma and to under 10% for the rectal carcinoma when consequently applying these principles of radicalness, as well as the 5-year survival could be increased up to 60.7% (3).

The conventional pre- and intra-operative staging studies do not allow to detect or predict synchronous or metachronous lymph node metastases in individual cases, respectively. Thus, a differentiation between stages I/II (UICC) and stage III is not possible before having performed the radical lymph node dissection in connection with a histopathological study of the resected tissue. As a result, identical principles of surgical radicalness actually apply for nearly all tumors of the clinical stages I-III.

Only in a sub-group of stage I (UICC) with small (≦3 cm) polypous pT1 carcinomas without lymph channel invasion (L0) and a lower or moderate degree of differentiation (G1/2), a lymphogenic metastasis (≦3%) can be excluded with adequate certainty due to conventional criteria of imaging and histopathology and a sole local resection (e.g. an endoscopic polypectomy, transrectal excision) can be performed (7).

A certain pre-operative exclusion of a lymphogenic metastasis would also allow the use of more gentle methods (e.g. endoscopic methods, laparoscopic tubular resections, combined endoscopic and laparoscopic methods, limited open resections) in further tumors in stage I and II (UICC). These little traumatizing invasions would enable an earlier mobilization of the patient and would involve a reduced hospitalization, a reduced need for analgetics and fewer complications during wound healing. Particularly, this would have very major advantages for older patients and patients at high risk for which the consequences of a radical resection are calculable to a lesser extent.

The actually available criteria for the prediction of the degree of differentiation, the infiltration depth and the lymph channel invasion are not sufficient to make a reliable pre-operative statement about the metastatic potential of colorectal carcinomas. The over-expression of MUC-1, ki67 or MMP-7 in immunohistochemical analyses of tumor tissue supposes a relation to a lymph node metastasis (8, 9, 10). Otherwise, these molecular markers could not be established as independent predictive factors for colorectal carcinomas, yet.

By using a micro-array analysis, genes could be identified in several studies which are specifically expressed in colorectal carcinomas or the healthy mucosa of the large intestine and thus allow a differentiation of both tissues (4, 5, 6).

So far, there are only insufficient studies existing in which it has been attempted to determine a predictive gene pattern for a metastasis in colorectal carcinomas. In the first study, the stages UICC II and III could be distinguished from each other via gene expression, however this was not the case for the stages UICC I and III. Furthermore, only 20 cases, each having 5 patients per stage UICC I-IV, had been included. One patient suffered from a familial tumor disease which is not acceptable for such a study, as gene expression can hereby be influenced. An early version of the GeneChip of the Affymetrix company having only 6,800 ProbeSets had been used (11). In a second micro-array study, only 12 patients had been included. Hereby, a cDNA micro-array having 4,608 ProbeSets manufactured for this purpose was used (12). In two additional studies concerning tumor progression, 25 cases were included, respectively. Here, also cases with liver metastasis (stage UICC IV) and even liver metastases themselves were included in the analyses. It has not been enlarged upon a lymph node metastasis (13-14). Due to the sample material's heterogeneity, the gene patterns listed in these papers hereto are questionable and do not show any correlation with the own gene pattern of the present work.

It is obvious that genes, which are specifically expressed in a particular stage of the colorectal carcinoma, can be involved in the pathologic specification of the respective stage. Hitherto, the patho-functions of stage-associated genes having been isolated by micro-array technology could not systematically be analyzed. This is due to the fact that there are generally identified gene groups (clusters) of genes, the expression of which is associated with a particular tumor stage, when performing comparing studies of gene expression with the micro-array technology. Additionally, combinational effects of these genes have to be considered for pathogenetic studies. However, this also explains why single genes could not have been established for predictive purposes yet. This concerns complex gene patterns for biological functions wherein the interactions of each single gene cause a lymph node metastasis.

Therefore, it is an object of the present invention to provide a micro-array for the prediction of a lymph node metastasis in connection with colorectal carcinoma and a respective ex vivo method showing an improved predictive value when compared to the test methods known so far. It is another object of the present invention to provide substances which are suitable for treating the metastasizing colorectal carcinoma.

These objects are solved by the subject-matter of the independent claims. Preferred aspects are given in the dependent claims.

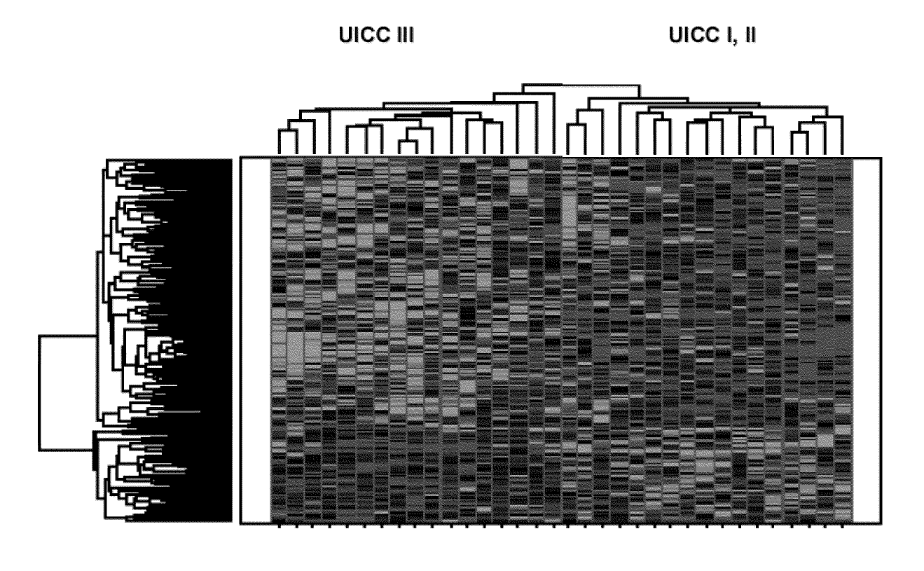

A predictive gene pattern of 240 genes for a lymph node metastasis could be detected by a micro-array analysis of 40 colorectal carcinomas without (stage UICC I, II) and 40 carcinomas with lymph node metastases (stage UICC III) in the present invention (table 1). The diligent selection of the cases and the exact histological control of the reprocessed tissues and a RNA control assure a corresponding data quality (15). Based on the present gene pattern, the prediction of a lymph node metastasis of 67% has been possible (FIG. 1), wherein the pre-operative radiological diagnostics according to the German Cancer Society Consensus Conference, Bochum, Germany, February 2004 (16), is only 60%. The collective studied represents the largest known number of cases used so far with a micro-array comprising about 22,280 ProbeSets (Affymetrix, HG-U133A). The detected gene set can, e.g., be used on a so called custom-made micro-array for pre-operative diagnostics on tumor biopsy after hybridization.

Tumor tissue of 40 patients with CRC stage UICC I, II and UICC III, respectively, has immediately been preserved post-operatively. After a macroscopic dissection for the enrichment with cancerous tissue, RNA has been isolated and hybridized on micro-arrays (HG-U133A, Affymetrix). PCR and immunohistochemical analyses have been performed on selected significantly differentially expressed genes.

The prediction for a LNM in connection with CRC is also possible in the biopsy of the primary tumor due to differentially expressed genes. This is the basis for individual pre-operative diagnostics and therapy planning.

Except for DCC and the micro-satellite instability, molecular predictive markers have not been established for clinical applications (see chapter “Examples”). Single markers are not able to predict a lymph node metastasis in the primary tumor. For the first time, a pattern of 240 predictive genes for a lymph node metastasis could have been evaluated in a sufficiently large random sample of 80 cases by using GenChip analyses (table 1). On this basis, a prediction for a lymph node metastasis can be carried out by means of a pre-operative biopsy of the primary tumor in future times. The composition of the genes as a specific pattern predicting the lymph node metastasis is hereby decisive (FIG. 1).

The present invention particularly comprises the following aspects and embodiments:

According to a first aspect, the invention relates to a micro-array for the prediction of a lymph node metastasis in connection with colorectal carcinoma wherein the micro-array comprises nucleic acids that are able to hybridize to at least a part of the subsequent nucleic acids or to their complementary or reverse or reverse-complementary sequences as well as to RNA sequences derived therefrom or to derivates of these sequences, wherein the nucleic acids at least comprise the nucleotide sequences according to table 2.

According to one embodiment of the invention, in addition to the afore-mentioned nucleic acids according to table 2, one or more of the nucleotide sequences according to table 1 are used in the micro-array. In other words, the micro-array according to the present invention is based at least on sequences according to table 2, but it can also be based on the sequences listed in table 1. In a preferred embodiment, the micro-array is based on all sequences according to table 1 (i.e. on all 240 sequences). It should be noted that table 1 comprises all sequences according to table 2. If the micro-array is thus based on more than the sequences according to table 2, these additional sequences will naturally be selected from the sequences according to table 1, which are not contained in table 2 yet.

The single sequences as well as their accession data in databases are detailed in table 1. As mentioned above, the gene composition is hereby decisive as being a specific pattern predicting the lymph node metastasis. The differential gene pattern between colorectal carcinomas without (stage UICC I, II) and with (stage UICC III) lymph node metastases can be seen from FIG. 1. These differences in the gene expression are used by the present invention in order to provide a micro-array and a diagnostic method having a high predictive value with respect to the prediction of a lymph node metastasis in connection with colorectal carcinoma.

As already mentioned above, in accordance with the present invention, particularly all 240 sequences from table 1 are consulted, it is, however, also possible, to limit the micro-array to sub-groups having an independent predictive value.

The nucleic acids of the present invention also comprise nucleic acids having sequences substantially being equivalent to the nucleic acids having SEQ ID NO: 1-240 (table 1). Nucleic acids according to the present invention can have, e.g., at least about 80%, typically at least about 90% or 95% sequence identity to the nucleic acids having SEQ ID NO: 1-240.

The term “micro-array” as used herein is a collective name for molecularbiological testing systems that enable the parallel analysis of up to several hundreds of single detections in a small amount of biological sample material. Special micro-arrays are sometimes also named “gene chips” or “biochips” as they contain numerous information in the smallest place.

The micro-arrays are primarily differed in DNA micro-arrays and protein micro-arrays.

Micro-arrays are used to detect the RNA amount of particular genes. There are mainly two different kinds of micro-arrays, on the one hand, there are micro-arrays based on bound cDNA and, on the other hand, there are micro-arrays based on synthetically produced oligonucleotides. These micro-arrays are used as probes which are applied on defined positions in a grid pattern on, e.g., glass carriers.

The following procedure is usually selected for the analysis:

At first, RNA is extracted from the patient's sample and transcribed in cDNA or cRNA after optional purification and/or amplification steps of the mRNA and are, for example, marked. Then, it is hybridized with the micro-arrays. In this way, marked cDNA/cRNA units bind to their complementary analoga on the array. After removing the unbound cDNA/cRNA units by washing, the signal of the marker is read by a suitable device in each position of the micro-array.

In context with the micro-arrays of the present invention, the “nucleic acids of the micro-array” are those sequences being part of the micro-array. They are basically complementary to the sequences of the nucleic acids having SEQ ID NO: 1-240 (being derived from patients' samples) and can, for example, consist of only a smaller segment of these sequences (for example in the case of oligonucleotide probes). Insofar, the term “nucleic acids specifically suited for hybridization” is used to describe those sequences bound to the micro-array which specifically bind to at least a segment of a corresponding nucleic acid sequence according to SEQ ID NO: 1-240.

According to a preferred embodiment, the nucleic acids having SEQ ID NO: 1-240 are chemically modified in order to enable/facilitate the readout/interpretation of the binding of the corresponding nucleic acids to the complementary nucleic acids of the micro-array. Preferably, there will be used a marker which is particularly selected from a radioactive marker, a fluorescence marker, a biotin marker, a digoxigenin marker, a peroxidase marker or a marker being detectable by alkaline phosphatase.

The nucleic acids of the micro-array are preferably bound to a solid phase matrix, e.g., a nylon membrane, glass or plastic material.

The micro-array is preferably a DNA, RNA or PNA array.

According to a second aspect, the present invention relates to an ex vivo method for predicting a lymph node metastasis in connection with colorectal carcinoma comprising the following steps:
taking a sample of the tumor tissue of the colorectal carcinoma from the patient;
extracting the RNA from the sample;
optionally transcribing the RNA in cDNA or cRNA;
detecting if the sample contains cDNA or cRNA which corresponds to the nucleic acid sequences according to SEQ ID NO: 1-240 or to one of the sub-groups thereof defined above.

According to a preferred embodiment, the afore-mentioned sub-groups are the nucleotide sequences according to table 2.

The detection of the nucleic acid sequences is preferably carried out by PCR, particularly by RT-PCR. The PCR method has the advantage that very small amounts of DNA can be detected. The temperature conditions and numbers of cycles have to be adapted depending on the material to be detected. The optimum reaction conditions can be determined in a well-known matter in manual experiments.

In the PCR reaction or the RT-PCR as well as in the hybridization reaction, the DNA or RNA, particularly the mRNA, of the sample to be assayed can either be present in an extracted form or in form of complex mixtures in which the DNA or RNA to be assayed only forms a very small part of the fraction of the special biological sample. The cells to be assayed can thus either be present in a purified form or, for example, be “contaminated” in a tissue assembly.

In the RT-PCR, the oligonucleotides of the invention are used for PCR amplification of fragments to cDNA templates being produced after a reverse transcription of the sample mRNA. The expression can then be evaluated qualitatively in combination with suited standards and techniques, like an internal standard and a quantitative PCR, also quantitatively.

Alternatively, the detection of the nucleic acids can be performed by complementary nucleic acid probes, preferably cDNA or oligonucleotide probes.

The nucleic acid probes are preferably able to hybridize to at least a segment of each nucleic acid or to complementary or reverse or reverse-complementary sequences thereof as well as to RNA sequences derived therefrom or derivatives of these sequences.

According to the present invention, hybridization is preferably carried out under moderately stringent or stringent conditions.

Stringent hybridization and washing conditions are generally understood as reaction conditions in which there are only produced duplex molecules between oligonucleotides and desired target molecules (perfect hybrids) or the desired target organism is detected, respectively. Stringent hybridization conditions are particularly understood as 0.2×SSC (0.03 M NaCl, 0.0003 M sodium citrate, pH 7) at 65° C. In shorter fragments, for example, oligonucleotides of up to 20 nucleotides, the hybridization temperature is below 65° C., for example, above 55° C., preferably above 60° C., but below 65° C., respectively. Stringent hybridization temperatures depend on the size or the length of the nucleic acid and its nucleotide composition, respectively, and have to be determined by a person skilled in the art by means of manual experiments. Moderately stringent conditions are, for example, reached at 42° C. and washing in 0.2×SSC/0.1% SDS at 42° C.

Alternatively, the respective nucleic acids can be detected by the proteins expressed therefrom, for example, by specific antibodies.

According to a third aspect, the present invention provides a method for detecting the expression of the genes specified in table 1 or 2, respectively, comprising the following steps:
extracting RNA from the patient's sample;
applying the RNA or nucleic acids derived therefrom on a micro-array as defined above; and
detecting the hybridization of the RNA or of nucleic acids derived therefrom to the nucleic acids of the micro-array.

As already described above, besides DNA arrays, there are protein micro-arrays which are also a subject-matter of the present invention. These are also used to predict a lymph node metastasis in connection with colorectal carcinoma, wherein the micro-array is able to detect the amino acid sequences corresponding to the nucleotide sequences of table 1 or 2.

According to a preferred embodiment, this is an antibody micro-array or a Western Blot micro-array. For example, in antibody micro-arrays, the antibodies are fixed (spotted) and then the sample is applied on the array. As it is used herein, the term “antibody” relates to intact antibodies as well as antibody fragments maintaining a special ability to selectively bind to an epitope. Those fragments comprise, without limitation, Fab, F(ab′)₂, recombinant single chain antibodies and Fv antibody fragments. The term “epitope” relates to any antigen determinant on an antigen onto which the paratope of an antibody binds. Epitope determinants are usually consisting of chemically active surface groups of molecules (for example, amino acid or sugar moieties) and typically have three-dimensional structural characteristics as well as special charge characteristics.

The antibodies according to the present invention can be produced by using any known method. For example, an amino acid or a fragment thereof expressed by a nucleic acid according to the present invention can be provided and used as an immunogen in order to evoke such an immune reaction in an animal in which specific antibodies will be produced.

The production of polyclonal antibodies is well-known to a person skilled in the art. See, for example, Green et al., Production of Polyclonal Antisera, in Immunochemical Protocols (Manson, eds.), pages 1-5 (Humana Press 1992) and Coligan et al., Production of Polyclonal Antisera in Rabbits, Rats, Mice and Hamsters, in Current Protocols in Immunology, chapter 2.4.1 (1992). Additionally, a person skilled in the art is aware of several techniques of immunology for purification and concentration of polyclonal antibodies as well as of monoclonal antibodies (Coligan et al., Unit 9, Current Protocols in Immunology, Wiley Interscience, 1994).

The production of monoclonal antibodies is also well-known to a person skilled in the art. See, for example, Köhler & Milstein, Nature 256: 495 (1975); Coligan et al., chapters 2.5.1-2.6.7; and Harlow et al., Antibodies: A Laboratory Manual, page 726 (Cold Spring Harbor Pub. 1988). Briefly, monoclonal antibodies can be extracted by injecting a composition comprising the amino acids according to the present invention into mice, then verifying the presence of an antibody production by examining a serum sample, removing the spleen for extracting B lymphocytes and fusing the B lymphocytes with myeloma cells for producing hybridomas, cloning the hybridomas, selecting the positive clones producing a monoclonal antibody against the protein and isolating the antibodies from the hybridoma cultures. Monoclonal antibodies can be isolated from hydridoma cultures by a number of well-established techniques and purified. Such isolation techniques include an affinity chromatography with protein A or G Sepharose, size exclusion chromatography and ion exchange chromatography. See, for example, Coligan et al., chapters 2.7.1-2.7.12 and chapter “Immunglobulin G (IgG)” in Methods In Molecular Biology, volume 10, pages 79-104 (Humana Press 1992).

The micro-array Western method is used to detect antigens in cell lysates of different tissues or in protein fractions extracted by isoelectric focussing. The cell lysate or the protein fraction is spotted on the carrier material of the micro-array, the antibody is applied subsequently. The antibody remains attached in each test panel showing antibody-antigen interaction. Panels with antibodies are then detected like in the Western Blot.

According to a further aspect, the invention relates to an inhibitor or a modulator of one or more sequences according to table 1 or 2 or of the proteins expressed therefrom. Such substances are used to treat colorectal carcinomas.

The invention also comprises a pharmaceutical composition comprising one or more of the inhibitors or modulators mentioned-above and a pharmaceutically acceptable carrier. Such a composition can (in addition to the active substances and the carrier) contain diluents, fillers, salts, buffers, stabilizers, solubilizers and other materials well-known in the art. The term “pharmaceutically acceptable” should define a non-toxic material which does not affect the efficiency of the biological activity of the active ingredient or the active substance, respectively. The selection of the carrier depends on the route of administration.

The pharmaceutical composition can additionally contain further agents enhancing the activity of the active substance or complementing the activity or use thereof during treatment (for example, chemotherapeutics). Such additional factors and/or agents can be contained in the pharmaceutical composition in order to achieve a synergistic effect or to minimize side effects or undesired effects, respectively. Techniques for formulating or preparing and administering the inhibitors/modulators of the present invention can be found in “Remington's Pharmaceutical Sciences”, Mack Publishing Co., Easton, Pa., last edition, respectively. A therapeutically effective dosage further refers to an amount of the compound sufficient to achieve an improvement of the symptoms, for example, a treatment, a healing, prevention or an improvement of such conditions. Suitable routes of administration can, for example, include oral, rectal, transmucosal or intestinal administration and parenteral administration, including intramuscular, subcutan, intramedular injections as well as intrathecal, directly intraventricular, intravenous, intraperitoneal or intranasal injections. The intravenous administration into a patient is preferred. The present invention will now be illustrated by the following examples and the FIGURE.

FIG. 1 shows a differential gene pattern between colorectal carcinomas without (stage UICC I, II) and with (stage UICC III) lymph node metastases. It shows a headmap for illustrating differentially expressed genes in stage UICC I, II vs. III. Differentially expressed genes are shown in the lines, red: over-expressed, green: under-expressed. The cases of the respective groups UICC I, II and III are shown in the columns. A pattern of differentially expressed genes UICC I, II vs. III can be clearly seen.

### EXAMPLES

The following summary shows predictive factors in connection with colorectal carcinoma:

Predictive factors in connection with colorectal carcinoma
(College of American Pathologists Consensus Statement 1999)

| Category I | Category IIA | | | |
|---|---|---|---|---|
| (statistically | (verifiably | | Category III | Category IV |
| detected explicit | predictive relevance, | Category IIB | (current data unclear/ | (verifiably no |
| predictive | clinical validation | (promising factors, | studies still | predictive |
| relevance) | to be proven) | data not sufficient) | insufficient) | relevance) |
| pT category | grading | histologic kind of | morphologic | tumor size |
| pN category | | carcinoma | marker: | |
| (AJCC/UICC) | | | perineural invasion | |
| | | | capillary density | |
| | | | (desmoplasia) | |
| | | | peritumoral | |
| | | | inflammatory | |
| | | | reaction | |
| | | | focal | |
| | | | neuroendocrine | |
| | | | differentiation | |
| V category | edges of the | MSI associated | all molecular | |
| (venous invasion) | resected tissue | factors, MSI-H | markers* except for | |
| L category | | | DCC and MSI-H, | |
| (lymph node | | | macroscopic tumor | |
| invasion) | | | configuration | |
| R classification | ypTNM | LOH 18q | all tumor markers* | |
| (residual tumor) | | (loss of DCC | (cell proteins and | |
| | | allele) | carbohydrates) | |
| | | | except for CEA | |
| CEA pre-operative | | configuration of | nuclear | |
| | | tumor edge | organisational | |
| | | | regions | |
| | | | proliferative | |
| | | | markers/index: Ki- | |
| | | | 67, DNA content | |

MSI: micro-satellite instability
CEA: carcinoma-embryonic antigen
ypTNM: y: classification according to radio/chemotherapy; p: pathologic staging
*marker list, see literatureAs specified above, FIG. 1 additionally shows a differential gene pattern between colorectal carcinomas without (stage UICC I, II) and with (stage UICC III) lymph node metastases, red: over-expressed genes, green: under-expressed genes.

The gene expression of 40 colorectal carcinomas without (stage UICC I and II) and of 40 colorectal carcinomas with lymph node metastases (stage UICC III) was compared in a study by using the Affymetrix GenChip technology. In this way, there could be detected 240 significantly differentially expressed genes UICC I, II vs. UICC III (see table 1). Based on these differentially expressed genes, there could be established a prediction for a lymph node metastasis of about 67% by several statistic models (Croner et al., Cancer 2005). Hitherto, a pre-operative statement about a lymph node metastasis has only been possible by means of imaging diagnostics. A realistic prediction of about 60% can be given herewith (German Cancer Society Consensus Conference, Bochum, Germany, February 2004). Single molecular markers could not be established as predictive relevant markers so far (FIG. 1).

With the present gene pattern, so-called “custom-made micro-arrays” (lymphchip) can be produced and used for pre-operative diagnostics. Colorectal carcinomas can then be biopsied pre-operatively. The isolated RNA from the tumor biopsies is hybridized to the lymphchip. A statement about a lymph node metastasis can be given depending on the gene expression pattern. On the basis of this information, an individual therapy with respect to operation planning or chemotherapy can be carried out.

TABLE 1
240 differentially expressed genes between colorectal carcinomas without
(stage UICC I, II) und with (stage UICC III) lymph node metastases.
The sequences correspond to SEQ ID NO: 1-240.

| GENBANK ® | | |
|---|---|---|
| accession number | description | P value |
| extracellular matrix | | |
| NM_021721 | a disintegrin and metalloproteinase domain 22 (ADAM22) | <0.001 |
| (SEQ ID NO: 1) | | |
| NM_024333 | fibronectin type III and SPRY domain containing 1 (FSD1) | <0.001 |
| (SEQ ID NO: 2) | | |
| NM_003613 | cartilage intermediate layer protein, nucleotide | <0.05 |
| (SEQ ID NO: 3) | pyrophosphohydrolase (CILP) | |
| NM_003635 | N-deacetylase/N-sulfotransferase (heparan glucosaminyl) 2 (NDST2) | <0.05 |
| (SEQ ID NO: 4) | | |
| cell cycle/mitosis | | |
| NM_000465 | BRCA1 associated RING domain 1 (BARD1) | <0.01 |
| (SEQ ID NO: 5) | | |
| X59065 | fibroblast growth factor 1 (acidic) (FGF1) | <0.01 |
| (SEQ ID NO: 6) | | |
| NM_018055 | nodal homolog (mouse) (NODAL) | <0.01 |
| (SEQ ID NO: 7) | | |
| NM_007370 | replication factor C (activator 1) 5, 36.5 kDa (RFC5) | <0.01 |
| (SEQ ID NO: 8) | | |
| U79302 | tumor protein p53 inducible protein 11 (TP53I11) | <0.05 |
| (SEQ ID NO: 155) | | |
| NM_022094 | cell death-inducing DFFA-like effector c (CIDEC) | <0.05 |
| (SEQ ID NO: 9) | | |
| BF967271 | anaphase promoting complex subunit 5 (ANAPC5) | <0.05 |
| (SEQ ID NO: 156) | | |
| NM_002634 | prohibitin (PHB) | <0.05 |
| (SEQ ID NO: 10) | | |
| AI367319 | SRY (sex determining region Y)-box 10 (SOX10) | <0.05 |
| (SEQ ID NO: 157) | | |
| NM_018165 | polybromo 1 (PB1) | <0.05 |
| (SEQ ID NO: 11) | | |
| AF074717 | RAD1 homolog (S. pombe) (RAD1) | <0.05 |
| (SEQ ID NO: 12) | | |
| NM_016567 | BRCA2 and CDKN1A interacting protein (BCCIP) | <0.05 |
| (SEQ ID NO: 13) | | |
| NM_004708 | programmed cell death 5 (PDCD5) | <0.05 |
| (SEQ ID NO: 14) | | |
| AF277724 | cell division cycle 25C (CDC25C) | <0.05 |
| (SEQ ID NO: 15) | | |
| AI042030 | SMC1 structural maintenance of chromosomes 1-like 1 (yeast) | <0.05 |
| (SEQ ID NO: 158) | (SMC1L1) | |
| AL161955 | triple functional domain (PTPRF interacting) (TRIO) | <0.05 |
| (SEQ ID NO: 159) | | |
| NM_006999 | polymerase (DNA directed) sigma (POLS) | <0.05 |
| (SEQ ID NO: 16) | | |
| NM_001786 | cell division cycle 2, G1 to S and G2 to M (CDC2) | <0.05 |
| (SEQ ID NO: 17) | | |
| NM_002898 | RNA binding motif, single stranded interacting protein 2 (RBMS2) | <0.05 |
| (SEQ ID NO: 18) | | |
| cell adhesion/ | | |
| cytoskeleton | | |
| NM_006262 | peripherin (PRPH) | <0.001 |
| (SEQ ID NO: 19) | | |
| AF312679 | Rhesus blood group, D antigen (RHD) | <0.001 |
| (SEQ ID NO: 20) | | |
| NM_001794 | cadherin 4, type 1, R-cadherin (retinal) (CDH4) | <0.01 |
| (SEQ ID NO: 21) | | |
| NM_030968 | C1q and tumor necrosis factor related protein 1 (C1QTNF1) | <0.01 |
| (SEQ ID NO: 23) | | |
| NM_012216 | midline 2 (MID2) | <0.01 |
| (SEQ ID NO: 24) | | |
| NM_020129 | lectin, galactoside-binding, soluble, 14 (LGALS14) | <0.01 |
| (SEQ ID NO: 25) | | |
| NM_001939 | dystrophin related protein 2 (DRP2) | <0.01 |
| (SEQ ID NO: 26) | | |
| AK001619 | phosphodiesterase 4D interacting protein (myomegalin) (PDE4DIP) | <0.01 |
| (SEQ ID NO: 160) | | |
| AW296788 | microtubule-associated protein 1A (MAP1A) | <0.01 |
| (SEQ ID NO: 161) | | |
| AF152503 | protocadherin gamma subfamily A, 10 (PCDH-gamma-A10) | <0.05 |
| (SEQ ID NO: 27) | | |
| NM_001232 | calsequestrin 2 (cardiac muscle) (CASQ2) | <0.05 |
| (SEQ ID NO: 28) | | |
| NM_000337 | sarcoglycan, delta (35 kDa dystrophin-associated glycoprotein) | <0.05 |
| (SEQ ID NO: 29) | (SGCD) | |
| NM_001944 | desmoglein 3 (pemphigus vulgaris antigen) (DSG3) | <0.05 |
| (SEQ ID NO: 30) | | |
| L20815 | corneodesmosin (CDSN) | <0.05 |
| (SEQ ID NO: 31) | | |
| NM_024883 | R-cadherin (retinal) (CDH4) | <0.015* |
| (SEQ ID NO: 240) | | |
| NM_006580 | claudin 16 (CLDN16) | <0.015* |
| (SEQ ID NO: 22) | | |
| channels/ | | |
| transporters | | |
| NM_020243 | translocase of outer mitochondrial membrane 22 homolog (yeast) | <0.01 |
| (SEQ ID NO: 32) | (TOMM22) | |
| N80922 | solute carrier family 35, member D1 (SLC35D1) | <0.05 |
| (SEQ ID NO: 162) | | |
| AF087138 | ATP-binding cassette, sub-family C (CFTR/MRP), member 8 | <0.05 |
| (SEQ ID NO: 163) | (ABCC8) | |
| NM_005895 | golgi autoantigen, golgin subfamily a, 3 (GOLGA3) | <0.05 |
| (SEQ ID NO: 33) | | |
| AW086154 | golgi associated, gamma adaptin ear containing, ARF binding protein | <0.05 |
| (SEQ ID NO: 164) | 2 (GGA2) | |
| U72069 | transportin 1 (TNPO1) | <0.05 |
| (SEQ ID NO: 165) | | |
| NM_024844 | pericentrin 1 (PCNT1) | <0.05 |
| (SEQ ID NO: 166) | | |
| AF047338 | solute carrier family 12, member 4 (SLC12A4) | <0.015* |
| (SEQ ID NO: 206) | | |
| AB046836 | transient receptor potential cation channel (TRPM3) | <0.015* |
| (SEQ ID NO: 207) | | |
| NM_007357 | component of oligomeric golgi complex 2 (COG2) | <0.015* |
| (SEQ ID NO: 208) | | |
| signal transduction | | |
| AA393484 | phospholipase C, beta 1 (phosphoinositide-specific) (PLCB1) | <0.001 |
| (SEQ ID NO: 167) | | |
| AY024365 | integrin-linked kinase-associated serine/threonine phosphatase 2C | <0.001 |
| (SEQ ID NO: 34) | (ILKAP) | |
| BE466525 | ecotropic viral integration site 1 (EVI1) | <0.001 |
| (SEQ ID NO: 168) | | |
| M15887 | diazepam binding inhibitor (DBI) | <0.001 |
| (SEQ ID NO: 35) | | |
| NM_030959 | olfactory receptor, family 12, subfamily D, member 3 (OR12D3) | <0.001 |
| (SEQ ID NO: 36) | | |
| NM_000541 | S-antigen, retina and pineal gland (arrestin) (SAG) | <0.01 |
| (SEQ ID NO: 37) | | |
| NM_014286 | frequenin homolog (Drosophila) (FREQ) | <0.01 |
| (SEQ ID NO: 38) | | |
| NM_016151 | TAO kinase 2 (TAOK2) | <0.01 |
| (SEQ ID NO: 39) | | |
| AF095723 | G protein-coupled receptors 51(GPR51) | <0.01 |
| (SEQ ID NO: 169) | | |
| AF015731 | glutamate receptor, ionotropic, N-methyl D-aspartate 1 (GRIN1) | <0.01 |
| (SEQ ID NO: 40) | | |
| AC004794 | olfactory receptor, family 1, subfamily I, member 1 (OR1I1) | <0.01 |
| (SEQ ID NO: 170) | | |
| AW138374 | Ras homolog enriched in brain (RHEB) | <0.01 |
| (SEQ ID NO: 171) | | |
| NM_002082 | G protein-coupled receptor kinase 6 (GRK6) | <0.01 |
| (SEQ ID NO: 41) | | |
| NM_016087 | wingless-type MMTV integration site family, member 16 (WNT16) | <0.05 |
| (SEQ ID NO: 42) | | |
| AI218134 | wingless-type MMTV integration site family, member 6 (WNT6) | <0.05 |
| (SEQ ID NO: 172) | | |
| AL050219 | immunoglobulin superfamily, member 4B (IGSF4B) | <0.05 |
| (SEQ ID NO: 173) | | |
| NM_003027 | SH3-domain GRB2-like 3 (SH3GL3) | <0.05 |
| (SEQ ID NO: 43) | | |
| M37981 | cholinergic receptor, nicotinic, alpha polypeptide 3 (CHRNA3) | <0.05 |
| (SEQ ID NO: 44) | | |
| AF229166 | docking protein 1, 62 kDa (downstream of tyrosine kinase 1) (DOK1) | <0.05 |
| (SEQ ID NO: 45) | | |
| NM_020548 | diazepam binding inhibitor (DBI) | <0.05 |
| (SEQ ID NO: 46) | | |
| AA459867 | olfactory receptor, family 7, subfamily E, member 156 pseudogene | <0.05 |
| (SEQ ID NO: 47) | (OR7E156P) | |
| AB000277 | discs, large (Drosophila) homolog-associated protein 1 (DLGAP1) | <0.05 |
| (SEQ ID NO: 48) | | |
| NM_021783 | ectodysplasin A2 receptor (EDA2R) | <0.05 |
| (SEQ ID NO: 49) | | |
| NM_012348 | olfactory receptor, family 2, subfamily N, member 1 pseudogene | <0.05 |
| (SEQ ID NO: 50) | (OR2N1P) | |
| NM_022644 | chorionic somatomammotropin hormone 2 (CSH2) | <0.05 |
| (SEQ ID NO: 51) | | |
| NM_016340 | Rap guanine nucleotide exchange factor (GEF) 6 (RAPGEF6) | <0.05 |
| (SEQ ID NO: 52) | | |
| NM_005754 | Ras-GTPase-activating protein SH3-domain-binding protein (G3BP) | <0.05 |
| (SEQ ID NO: 53) | | |
| AW192876 | casein kinase 1, epsilon (CSNK1E) | <0.05 |
| (SEQ ID NO: 54) | | |
| NM_000529 | melanocortin 2 receptor (adrenocorticotropic hormone) (MC2R) | <0.05 |
| (SEQ ID NO: 55) | | |
| AK022494 | RAB3 GTPase-activating protein (RAB3GAP) | <0.05 |
| (SEQ ID NO: 174) | | |
| NM_003696 | olfactory receptor, family 6, subfamily A, member 2 (OR6A2) | <0.05 |
| (SEQ ID NO: 56) | | |
| NM_005374 | membrane protein, palmitoylated 2 (MPP2) | <0.015* |
| (SEQ ID NO: 209) | | |
| NM_001883 | corticotropin releasing hormone receptor 2 (CRHR2) | <0.015* |
| (SEQ ID NO: 210) | | |
| NM_022559 | growth hormone 1 (GH1) | <0.015* |
| (SEQ ID NO: 211) | | |
| M95489 | follicle stimulating hormone receptor (FSHR) | <0.015* |
| (SEQ ID NO: 212) | | |
| metabolism | | |
| NM_014222 | NADH dehydrogenase (ubiquinone) 1 alpha subcomplex, 8, 19 kDa | <0.001 |
| (SEQ ID NO: 57) | (NDUFA8) | |
| AI252582 | ATPase, H+ transporting, lysosomal 9 kDa, V0 subunit (ATP6V0E) | <0.001 |
| (SEQ ID NO: 175) | | |
| M29873 | cytochrome P450, family 2, subfamily B, polypeptide 7 pseudogene 1 | <0.001 |
| (SEQ ID NO: 58) | (CYP2B7P1) | |
| NM_000284 | pyruvate dehydrogenase (lipoamide) alpha 1 (PDHA1) | <0.01 |
| (SEQ ID NO: 59) | | |
| AF000381 | folate receptor 1 (adult) (FOLR1) | <0.01 |
| (SEQ ID NO: 60) | | |
| AW024233 | glycine-N-acyltransferase (GLYAT) | <0.01 |
| (SEQ ID NO: 61) | | |
| M33318 | cytochrome P450, family 2, subfamily A, polypeptide 6 (CYP2A6) | <0.01 |
| (SEQ ID NO: 62) | | |
| Z97630 | glycine C-acetyltransferase (2-amino-3-ketobutyrate coenzyme A | <0.01 |
| (SEQ ID NO: 176) | ligase) (GCAT) | |
| NM_001740 | calbindin 2, 29 kDa (calretinin) (CALB2) | <0.01 |
| (SEQ ID NO: 63) | | |
| D13119 | ATP synthase, H+ transporting, mitochondrial F0 complex, subunit c | <0.05 |
| (SEQ ID NO: 64) | (ATP5G2) | |
| AA669797 | fumarate hydratase (FH), bone marrow | <0.05 |
| (SEQ ID NO: 65) | | |
| NM_000143 | fumarate hydratase (FH), mitochondrial | <0.05 |
| (SEQ ID NO: 66) | | |
| BC005270 | NADH dehydrogenase (ubiquinone) Fe—S protein 4, 18 kDa | <0.05 |
| (SEQ ID NO: 67) | (NDUFS4) | |
| AB018272 | ubiquitin specific protease 34 (USP34) | <0.05 |
| (SEQ ID NO: 177) | | |
| J04178 | hexosaminidase A (alpha polypeptide) (HEXA) | <0.05 |
| (SEQ ID NO: 68) | | |
| NM_005917 | malate dehydrogenase 1, NAD (soluble) (MDH1) | <0.05 |
| (SEQ ID NO: 69) | | |
| NM_001190 | branched chain aminotransferase 2, mitochondrial (BCAT2) | <0.05 |
| (SEQ ID NO: 70) | | |
| AY028632 | catalase (CAT) | <0.05 |
| (SEQ ID NO: 71) | | |
| NM_003000 | succinate dehydrogenase complex, subunit B, iron sulfur (Ip) | <0.05 |
| (SEQ ID NO: 72) | (SDHB) | |
| NM_001869 | carboxypeptidase A2 (pancreatic) (CPA2) | <0.05 |
| (SEQ ID NO: 73) | | |
| NM_004547 | NADH dehydrogenase (ubiquinone) 1 beta subcomplex, 4, 15 kDa | <0.05 |
| (SEQ ID NO: 74) | (NDUFB4) | |
| NM_000281 | 6-pyruvoyl-tetrahydropterin synthase (PCBD) | <0.05 |
| (SEQ ID NO: 75) | | |
| N71116 | phospholipase A2, group IVB (cytosolic) (PLA2G4B) | <0.05 |
| (SEQ ID NO: 178) | | |
| NM_000818 | glutamate decarboxylase 2 (pancreatic islets and brain, 65 kDa) | <0.05 |
| (SEQ ID NO: 76) | (GAD2) | |
| AF280110 | cytochrome P450, family 3, subfamily A, polypeptide 43 (CYP3A43) | <0.05 |
| (SEQ ID NO: 77) | | |
| BC006229 | cytochrome c oxidase subunit Vb (COX5B) | <0.05 |
| (SEQ ID NO: 78) | | |
| M64992 | proteasome (prosome, macropain) subunit, alpha type, 1 (PSMA1) | <0.05 |
| (SEQ ID NO: 79) | | |
| NM_000055 | butyrylcholinesterase (BCHE) | <0.015* |
| (SEQ ID NO: 213) | | |
| miscellaneous | | |
| NM_014814 | proteasome regulatory particle subunit p44S10 (PSMD6) | <0.001 |
| (SEQ ID NO: 80) | | |
| NM_001189 | bagpipe homeobox homolog 1 (Drosophila) (BAPX1) | <0.001 |
| (SEQ ID NO: 81) | | |
| NM_006839 | inner membrane protein, mitochondrial (mitofilin) (IMMT) | <0.001 |
| (SEQ ID NO: 82) | | |
| AI278616 | SET translocation (myeloid leukemia-associated) (SET) | <0.01 |
| (SEQ ID NO: 83) | | |
| NM_003171 | suppressor of var1, 3-like 1 (S. cerevisiae) (SUPV3L1) | <0.01 |
| (SEQ ID NO: 84) | | |
| NM_006604 | ret finger protein-like 3 (RFPL3) | <0.01 |
| (SEQ ID NO: 85) | | |
| NM_004960 | fusion (involved in t(12, 16) in malignant liposarcoma) (FUS) | <0.01 |
| (SEQ ID NO: 86) | | |
| AK026273 | FK506 binding protein 1B (FKBP1B) | <0.01 |
| (SEQ ID NO: 179) | | |
| NM_007011 | abhydrolase domain containing 2 (ABHD2) | <0.01 |
| (SEQ ID NO: 87) | | |
| BC006466 | diazepam binding inhibitor (DBI) | <0.01 |
| (SEQ ID NO: 88) | | |
| NM_019009 | toll interacting protein (TOLLIP) | <0.05 |
| (SEQ ID NO: 89) | | |
| NM_021922 | Fanconi anemia, complementation group E (FANCE) | <0.05 |
| (SEQ ID NO: 90) | | |
| AL581473 | exosome component 7 (EXOSC7) | <0.05 |
| (SEQ ID NO: 180) | | |
| NM_012123 | mitochondrial translation optimization 1 homolog (S. cerevisiae) | <0.05 |
| (SEQ ID NO: 91) | (MTO1) | |
| BC003170 | ubiquitin associated protein 2-like (UBAP2L) | <0.05 |
| (SEQ ID NO: 92) | | |
| AF073483 | basophilic leukemia expressed protein BLES03 (Bles03) | <0.05 |
| (SEQ ID NO: 181) | | |
| U13395 | WW domain containing oxidoreductase (WWOX) | <0.05 |
| (SEQ ID NO: 93) | | |
| X55503 | metallothionein IV (MT4) | <0.05 |
| (SEQ ID NO: 94) | | |
| AK024944 | mediator of RNA polymerase II transcription, subunit 28 homolog | <0.05 |
| (SEQ ID NO: 182) | (MED28) | |
| AF263541 | dual-specificity tyrosine-(Y)-phosphorylation regulated kinase 4 | <0.05 |
| (SEQ ID NO: 95) | (DYRK4) | |
| U67122 | SMT3 suppressor of mif two 3 homolog 1 (yeast) (SUMO1) | <0.05 |
| (SEQ ID NO: 96) | | |
| NM_018234 | dudulin 2 (TSAP6) | <0.05 |
| (SEQ ID NO: 183) | | |
| NM_003905 | amyloid beta precursor protein binding protein 1, 59 kDa (APPBP1) | <0.05 |
| (SEQ ID NO: 97) | | |
| AK024527 | FLJ20874 fis, clone ADKA02818 | <0.05 |
| (SEQ ID NO: 98) | | |
| AF168617 | sperm associated antigen 11 (SPAG11) | <0.015* |
| (SEQ ID NO: 214) | | |
| BC006333 | tripartite motif-containing 14 (TRIM14) | <0.015* |
| (SEQ ID NO: 215) | | |
| NM_007009 | zona pellucida binding protein (ZPBP) | <0.015* |
| (SEQ ID NO: 216) | | |
| NM_014420 | dickkopf (Xenopus laevis) homolog 4 (DKK4) | <0.01 |
| (SEQ ID NO: 217) | | |
| X98568 | Type X collagen gene | <0.01 |
| (SEQ ID NO: 218) | | |
| BC000340 | melanoma antigen, family A, 3, clone MGC: 8564 | <0.01 |
| (SEQ ID NO: 219) | | |
| NM_005940 | matrix metalloproteinase 11 (stromelysin 3) (MMP11) | <0.01 |
| (SEQ ID NO: 220) | | |
| NM_013307 | non-functional folate binding protein (HSAF000381) | <0.01 |
| (SEQ ID NO: 221) | | |
| NM_018678 | lipopolysaccharide specific response-68 protein (LSR68) | <0.01 |
| (SEQ ID NO: 222) | | |
| AF036973 | HLA complex group 4 pseudogene 6 (HCG4P6) | <0.015* |
| (SEQ ID NO: 223) | | |
| function unknown | | |
| H61826 | Tularik gene 1, NIK and IKK binding protein (NIBP) | <0.001 |
| (SEQ ID NO: 184) | | |
| BF223370 | hypothetical protein MGC11332 (MGC11332) | <0.01 |
| (SEQ ID NO: 99) | | |
| N64803 | trinucleotide repeat containing 6B (TNRC6B) | <0.01 |
| (SEQ ID NO: 185) | | |
| NM_016458 | chromosome 8 open reading frame 30A (LOC51236) | <0.01 |
| (SEQ ID NO: 186) | | |
| BF055496 | DKFZP586J0619 protein (DKFZP586J0619) | <0.01 |
| (SEQ ID NO: 187) | | |
| NM_004939 | DEAD (Asp-Glu-Ala-Asp) box polypeptide 1 (DDX1) | <0.01 |
| (SEQ ID NO: 100) | | |
| AK001854 | KIAA0931 protein (PHLPPL) | <0.01 |
| (SEQ ID NO: 188) | | |
| AL137303 | KIAA1055 protein (KIAA1055) | <0.01 |
| (SEQ ID NO: 189) | | |
| AW014299 | hypothetical protein MGC13098 (MGC13098) | <0.01 |
| (SEQ ID NO: 101) | | |
| AW025284 | hypothetical protein LOC222070 (LOC222070) | <0.01 |
| (SEQ ID NO: 102) | | |
| AK025343 | hepatocellular carcinoma-related HCRP1 (HCRP1) | <0.01 |
| (SEQ ID NO: 190) | | |
| AB023170 | KIAA0953 (KIAA0953) | <0.05 |
| (SEQ ID NO: 103) | | |
| AK027146 | hypothetical LOC388650 (LOC388650) | <0.05 |
| (SEQ ID NO: 104) | | |
| AK025833 | CD33 antigen-like 3 (CD33L3) | <0.05 |
| (SEQ ID NO: 191) | | |
| NM_018253 | YY1 associated protein (YAP) | <0.05 |
| (SEQ ID NO: 105) | | |
| AI431902 | hypothetical protein FLJ13491 (FLJ13491) | <0.05 |
| (SEQ ID NO: 106) | | |
| AK022688 | KIAA0863 protein (KIAA0863) | <0.05 |
| (SEQ ID NO: 192) | | |
| AK026465 | FLJ22812 fis (FLJ22812) | <0.05 |
| (SEQ ID NO: 107) | | |
| NM_017700 | hypothetical protein FLJ20184 | <0.05 |
| (SEQ ID NO: 108) | | |
| NM_024519 | hypothetical protein FLJ13725 | <0.05 |
| (SEQ ID NO: 193) | | |
| NM_024035 | hypothetical protein MGC3113 | <0.05 |
| (SEQ ID NO: 109) | | |
| U72513 | RPL13-2 pseudogene (LOC283345) | <0.05 |
| (SEQ ID NO: 110) | | |
| BF436315 | Nedd4 binding protein 1 (N4BP1) | <0.05 |
| (SEQ ID NO: 194) | | |
| AL031228 | DNA segment on chromosome 6 (D6S2723E) | <0.05 |
| (SEQ ID NO: 111) | | |
| NM_018053 | hypothetical protein FLJ10307 (FLJ10307) | <0.05 |
| (SEQ ID NO: 195) | | |
| AF334946 | FKSG44 gene (FKSG44) | <0.05 |
| (SEQ ID NO: 112) | | |
| NM_022371 | torsin family 3, member A (TOR3A) | <0.05 |
| (SEQ ID NO: 113) | | |
| NM_004209 | synaptogyrin 3 (SYNGR3) | <0.05 |
| (SEQ ID NO: 114) | | |
| AF052091 | BTB (POZ) domain containing 9 (BTBD9) | <0.05 |
| (SEQ ID NO: 196) | | |
| AK000834 | FLJ20827 fis (FLJ20827) | <0.05 |
| (SEQ ID NO: 115) | | |
| AL022101 | hypothetical protein LOC343070 (LOC343070) | <0.05 |
| (SEQ ID NO: 116) | | |
| U79458 | WW domain binding protein 2 (WBP2) | <0.05 |
| (SEQ ID NO: 117) | | |
| BC005078 | hypothetical protein FLJ10996 (FLJ10996) | <0.05 |
| (SEQ ID NO: 118) | | |
| NM_025125 | chromosome 10 open reading frame 57 (C10orf57) | <0.05 |
| (SEQ ID NO: 119) | | |
| AF225421 | HT017 protein (HT017) | <0.05 |
| (SEQ ID NO: 120) | | |
| AL050370 | hypothetical protein FLJ33790 (FLJ33790) | <0.05 |
| (SEQ ID NO: 121) | | |
| AB051441 | KIAA1654 protein (KIAA1654) | <0.05 |
| (SEQ ID NO: 122) | | |
| AB051447 | KIAA1660 protein (KIAA1660) | <0.05 |
| (SEQ ID NO: 123) | | |
| NM_024037 | hypothetical protein MGC2603 (MGC2603) | <0.05 |
| (SEQ ID NO: 197) | | |
| AL024509 | 60S Ribosomal Protein L21 pseudogene (HNRPA3) | <0.05 |
| (SEQ ID NO: 124) | | |
| X72882 | KIAA0409 protein (KIAA0409) | <0.05 |
| (SEQ ID NO: 198) | | |
| AL137435 | hypothetical gene carriered by AL137435 (LOC142779) | <0.05 |
| (SEQ ID NO: 125) | | |
| AW003733 | Ras homolog gene family, member D (RHOD) | <0.05 |
| (SEQ ID NO: 126) | | |
| AL137475 | DKFZp434F0723 protein (DKFZp434F0723) | <0.05 |
| (SEQ ID NO: 127) | | |
| NM_025094 | hypothetical protein FLJ22184 (FLJ22184) | <0.05 |
| (SEQ ID NO: 128) | | |
| NM_025052 | Yeast Sps1/Ste20-related kinase 4 (YSK4) | <0.05 |
| (SEQ ID NO: 129) | | |
| NM_006010 | arginine-rich, mutated in early stage tumors (ARMET) | <0.05 |
| (SEQ ID NO: 130) | | |
| AK025127 | SATB family member 2 (SATB2) | <0.05 |
| (SEQ ID NO: 199) | | |
| AK024527 | FLJ20874 fis, clone ADKA02818 (AK024527) | <0.05 |
| (SEQ ID NO: 98) | | |
| NM_014118 | Glycosyltransferase-like domain containing 1 (GTDC1) | <0.05 |
| (SEQ ID NO: 131) | | |
| BC000978 | FLJ20259 (FLJ20259) | <0.015* |
| (SEQ ID NO: 224) | | |
| N55205 | hemoglobin, beta pseudogene 1 (HBBP1) | <0.015* |
| (SEQ ID NO: 225) | | |
| NM_024748 | FLJ11539 (FLJ11539) | <0.015* |
| (SEQ ID NO: 226) | | |
| AF288389 | glycosyltransferase 25 domain containing 2 (GLT25D2) | <0.015* |
| (SEQ ID NO: 227) | | |
| AL121891 | U-box domain containing 5 (UBOX5) | <0.015* |
| (SEQ ID NO: 228) | | |
| AF070610 | clone 24505 (BEAN) | <0.015* |
| (SEQ ID NO: 229) | | |
| D85939 | craniofacial development protein 1 (CFDP1) | <0.015* |
| (SEQ ID NO: 230) | | |
| D38081 | thromboxane A2 receptor (TBXA2R) | <0.015* |
| (SEQ ID NO: 231) | | |
| AI263044 | qz29e03.x1 NCI_CGAP_Kid11 cDNA clone | <0.015* |
| (SEQ ID NO: 232) | | |
| NM_014772 | KIAA0427 (KIAA0427) | <0.015* |
| (SEQ ID NO: 233) | | |
| U85995 | parathyroid hormone-responsive B1 (PTHB1) | <0.015* |
| (SEQ ID NO: 234) | | |
| AW975818 | YTH domain containing 2 (YTHDC2) | <0.015* |
| (SEQ ID NO: 235) | | |
| NM_024593 | EF-hand calcium binding domain 1 (EFCAB1) | <0.015* |
| (SEQ ID NO: 236) | | |
| BF968960 | TM2 domain containing 1 (TM2D1) | <0.015* |
| (SEQ ID NO: 237) | | |
| AF151022 | HSPC 188 | <0.015* |
| (SEQ ID NO: 238) | | |
| protein synthesis | | |
| AF279891 | DEAH (Asp-Glu-Ala-His) box polypeptide 15 (DHX15) | <0.001 |
| (SEQ ID NO: 132) | | |
| D21851 | leucyl-tRNA synthetase 2, mitochondrial (LARS2) | <0.001 |
| (SEQ ID NO: 200) | | |
| NM_002372 | mannosidase, alpha, class 2A, member 1 (MAN2A1) | <0.01 |
| (SEQ ID NO: 133) | | |
| NM_007362 | nuclear cap binding protein subunit 2, 20 kDa (NCBP2) | <0.01 |
| (SEQ ID NO: 134) | | |
| NM_022163 | mitochondrial ribosomal protein L46 (MRPL46) | <0.05 |
| (SEQ ID NO: 135) | | |
| AW118072 | signal recognition particle receptor, B subunit (SRPRB) | <0.05 |
| (SEQ ID NO: 201) | | |
| NM_005826 | heterogeneous nuclear ribonucleoprotein R (HNRPR) | <0.05 |
| (SEQ ID NO: 136) | | |
| BF195526 | heterogeneous nuclear ribonucleoprotein A3 (HNRPA3P1) | <0.05 |
| (SEQ ID NO: 202) | | |
| BF033354 | splicing factor, arginine/serine-rich 7, 35 kDa (SFRS7) | <0.05 |
| (SEQ ID NO: 203) | | |
| NM_001029 | ribosomal protein S26 (RPS26) | <0.05 |
| (SEQ ID NO: 137) | | |
| NM_001357 | DEAH (Asp-Glu-Ala-His) box polypeptide 9 (DHX9) | <0.05 |
| (SEQ ID NO: 138) | | |
| NM_020191 | mitochondrial ribosomal protein S22 (MRPS22) | <0.05 |
| (SEQ ID NO: 139) | | |
| NM_013417 | isoleucine-tRNA synthetase (LARS) | <0.05 |
| (SEQ ID NO: 140) | | |
| U37689 | polymerase (RNA) II (DNA directed) polypeptide H (POLR2H) | <0.05 |
| (SEQ ID NO: 141) | | |
| gene control | | |
| NM_012317 | leucine zipper, down-regulated in cancer 1 (LDOC1) | <0.001 |
| (SEQ ID NO: 142) | | |
| NM_002967 | scaffold attachment factor B (SAFB) | <0.01 |
| (SEQ ID NO: 143) | | |
| NM_004821 | heart and neural crest derivatives expressed 1 (BARD1) | <0.01 |
| (SEQ ID NO: 144) | | |
| NM_006365 | transcriptional activator of the c-fos promoter (CROC4) | <0.01 |
| (SEQ ID NO: 204) | | |
| BC002818 | synovial sarcoma, X breakpoint 2 (SSX2) | <0.01 |
| (SEQ ID NO: 145) | | |
| NM_014910 | zinc finger protein 507 (ZNF507) | <0.01 |
| (SEQ ID NO: 146) | | |
| AL080144 | ELYS transcription factor-like protein TMBS62 (ELYS) | <0.05 |
| (SEQ ID NO: 205) | | |
| M92439 | leucine-rich PPR-motif containing (LRPPRC) | <0.05 |
| (SEQ ID NO: 147) | | |
| NM_006963 | zinc finger protein 22 (KOX 15) (ZNF22) | <0.05 |
| (SEQ ID NO: 148) | | |
| NM_003927 | methyl-CpG binding domain protein 2 (MBD2) | <0.05 |
| (SEQ ID NO: 149) | | |
| BF530257 | transcriptional activator of the c-fos promoter (CROC4) | <0.05 |
| (SEQ ID NO: 150) | | |
| NM_001189 | bagpipe homeobox homolog 1 (Drosophila) (BAPX1) | <0.015* |
| (SEQ ID NO: 81) | | |
| BC001205 | SIN3 homolog B, transcription regulator (yeast) (SIN3B) | <0.015* |
| (SEQ ID NO: 239) | | |
| immune system | | |
| AF047245 | immunoglobulin lambda locus (IGL) | <0.01 |
| (SEQ ID NO: 151) | | |
| NM_020393 | peptidoglycan recognition protein 4 (PGLYRP4) | <0.01 |
| (SEQ ID NO: 152) | | |
| X95660 | immunoglobulin heavy constant mu (IGHM) | <0.05 |
| (SEQ ID NO: 153) | | |
| AI659611 | inducible T-cell co-stimulator ligand (ICOSL) | <0.05 |
| (SEQ ID NO: 154) | | |

*based on multiple tests on all 22,286 ProbeSets GenChips (Affymetrix HG-U133A).

TABLE 2
a sub-group of 50 nucleic acids reaching a high predictive value in the prediction
of a lymph node metastasis in connection with colorectal carcinoma. p < 0.015
based on multiple tests on all 22,286 ProbeSets GenChips (Affymetrix HG-U133A).

| GENBANK ® | |
|---|---|
| accession number | description |
| cell adhesion/cytoskeleton | |
| NM_021721 | ADAM metallopeptidase domain 22 (ADAM22) |
| (SEQ ID NO: 1) | |
| NM_006262 | peripherin (PRPH) |
| (SEQ ID NO: 19) | |
| NM_024883 | R-cadherin (retinal) (CDH4) |
| (SEQ ID NO: 240) | |
| NM_006580 | claudin 16 (CLDN16) |
| (SEQ ID NO: 22) | |
| transporters/channels | |
| N80922 | Solute carrier family 35, member D1 (SLC35D1) |
| (SEQ ID NO: 162) | |
| AI252582 | ATPase, H+ transporting, lysosomal 9 kDa, V0 subunit (ATP6V0E) |
| (SEQ ID NO: 175) | |
| AF047338 | Solute carrier family 12, member 4 (SLC12A4) |
| (SEQ ID NO: 206) | |
| AB046836 | transient receptor potential cation channel, subfamily M, member 3 (TRPM3) |
| (SEQ ID NO: 207) | |
| NM_007357 | component of oligomeric golgi complex 2 (COG2) |
| (SEQ ID NO: 208) | |
| signal transduction | |
| H61826 | NIK and IKK{beta} binding protein (NIBP) |
| (SEQ ID NO: 184) | |
| NM_030959 | olfactory receptor, family 12, subfamily D, member 3 (OR12D3) |
| (SEQ ID NO: 36) | |
| BE466525 | ecotropic viral integration site 1 (EVI1) |
| (SEQ ID NO: 168) | |
| NM_016087 | wingless-type MMTV integration site family, member 16 (WNT16) |
| (SEQ ID NO: 42) | |
| M95489 | follicle stimulating hormone receptor (FSHR) |
| (SEQ ID NO: 212) | |
| NM_005374 | membrane protein, palmitoylated 2 (MAGUK p55 subfamily member 2) (MPP2) |
| (SEQ ID NO: 209) | |
| NM_001883 | corticotropin releasing hormone receptor 2 (CRHR2) |
| (SEQ ID NO: 210) | |
| NM_022559 | growth hormone 1 (GH1) |
| (SEQ ID NO: 211) | |
| D38081 | thromboxane A2 receptor (TBXA2R) |
| (SEQ ID NO: 231) | |
| metabolism | |
| NM_014222 | NADH dehydrogenase (ubiquinone) 1 alpha subcomplex, 8, 19 kDa (NDUFA8) |
| (SEQ ID NO: 57) | |
| AW024233 | glycine-N-acyltransferase (GLYAT) |
| (SEQ ID NO: 61) | |
| NM_014814 | proteasome (prosome, macropain) 26S subunit, non-ATPase, 6 (PSMD6) |
| (SEQ ID NO: 80) | |
| NM_000055 | butyrylcholinesterase (BCHE) |
| (SEQ ID NO: 213) | |
| protein synthesis | |
| AF279891 | DEAH (Asp-Glu-Ala-His) box polypeptide 15 (DHX15) |
| (SEQ ID NO: 132) | |
| D21851 | leucyl-tRNA synthetase 2, mitochondrial (LARS2) |
| (SEQ ID NO: 200) | |
| gene control | |
| NM_001189 | bagpipe homeobox homolog 1 (Drosophila) (BAPX1) |
| (SEQ ID NO: 81) | |
| BC001205 | SIN3 homolog B, transcription regulator (yeast) (SIN3B) |
| (SEQ ID NO: 239) | |
| immune system | |
| NM_020393 | peptidoglycan recognition protein 4 (PGLYRP4) |
| (SEQ ID NO: 152) | |
| miscellaneous | |
| AF168617 | sperm associated antigen 11 (SPAG11) |
| (SEQ ID NO: 214) | |
| BC006333 | tripartite motif-containing 14 (TRIM14) |
| (SEQ ID NO: 215) | |
| NM_007009 | zona pellucida binding protein (ZPBP) |
| (SEQ ID NO: 216) | |
| M29873 | cytochrome P450, family 2, subfamily B, polypeptide 7 pseudogene 1 |
| (SEQ ID NO: 58) | (CYP2B7P1) |
| N55205 | hemoglobin, beta pseudogene 1 (HBBP1) |
| (SEQ ID NO: 225) | |
| AF036973 | HLA complex group 4 pseudogene 6 (HCG4P6) |
| (SEQ ID NO: 223) | |
| function unknown | |
| N64803 | trinucleotide repeat containing 6B (TNRC6B) |
| (SEQ ID NO: 185) | |
| NM_016458 | chromosome 8 open reading frame 30A (LOC51236) |
| (SEQ ID NO: 186) | |
| NM_006604 | ret finger protein-like 3 (RFPL3) |
| (SEQ ID NO: 85) | |
| NM_024333 | fibronectin type III and SPRY domain containing 1 (FSD1) |
| (SEQ ID NO: 2) | |
| BC000978 | FLJ20259 (FLJ20259) |
| (SEQ ID NO: 224) | |
| NM_024748 | FLJ11539 (FLJ11539) |
| (SEQ ID NO: 226) | |
| AF288389 | glycosyltransferase 25 domain containing 2 (GLT25D2) |
| (SEQ ID NO: 227) | |
| AL121891 | U-box domain containing 5 (UBOX5) |
| (SEQ ID NO: 228) | |
| AF070610 | Clone 24505 (BEAN) |
| (SEQ ID NO: 229) | |
| D85939 | craniofacial development protein 1 (CFDP1) |
| (SEQ ID NO: 230) | |
| AI263044 | qz29e03.x1 NCI_CGAP_Kid11 cDNA clone |
| (SEQ ID NO: 232) | |
| NM_014772 | KIAA0427 (KIAA0427) |
| (SEQ ID NO: 233) | |
| U85995 | parathyroid hormone-responsive B1 (PTHB1) |
| (SEQ ID NO: 234) | |
| AW975818 | YTH domain containing 2 (YTHDC2) |
| (SEQ ID NO: 235) | |
| NM_024593 | EF-hand calcium binding domain 1 (EFCAB1) |
| (SEQ ID NO: 236) | |
| BF968960 | TM2 domain containing 1 (TM2D1) |
| (SEQ ID NO: 237) | |
| AF151022 | HSPC 188 |
| (SEQ ID NO: 238) | |

A list of the sequences of the sequences listed in table 1 and 2 is given in the Sequence Listing. All sequences are derived from humans (Homo sapiens).

### LITERATURE

1. Hohenberger W: The effect of specialization or organisation of rectal cancer surgery; in: Rectal Cancer Surgery, Optimisation, Standardisation, Documentation. Springer Verlag Berlin Heidelberg, Soreide O, Norstein J (eds) pp 353-363 (1997)
2. Hohenberger W. Schick Ch, Göhl J: Mesorectal lymph node dissection: is it beneficial? Langenbeck's Arch Surg 383: 402-408 (1998)
3. Hermanek P. Wichelt H. Staimmer D, Riedl St. and the German Study Group Colo-Rectal Carcinoma (SGCRC): Prognostic factors of rectum carcinoma—experience of the German multicentre study SGCRC Tumori 81 Supplement: 60-64; (1995)
4. Birkenkamp-Demtroder, K., L. L. Christensen, et al. (2002). “Gene expression in colorectal cancer.” Cancer Res 62: 4352-63.
5. Notterman, D. A., U. Alon, et al. (2001). “Transcriptional gene expression profiles of colorectal adenoma, adenocarcinoma, and normal tissue examined by oligonucleotide arrays.” Cancer Res 61: 3124-30.
6. Croner R S, Foertsch T, Brueckl W M, Guenther K, Siebenhaar R, Matzel K E, Kirchner T, Behrens J, Klein-Hitpass L, Stuerzl M, Hohenberger W and Reingruber B. Common denominator genes that distinguish colorectal mucosa from carcinoma. Int J Colorectal Disease, Epub ahead of print 22.12.2004
7. S. Merkel, C. Rödel, W. Hohenberger. Colorectal Carcinoma-Surgical treatment. Endo heute 2004; 17:97-103.
8. Tanaka S, Haruma K, Tatsuta S, Hiraga Y, Teixeira C R, Shimamoto F, Yoshihara M, Sumii K, Kajiyama G. Proliferating cell nuclear antigen expression correlates with the metastatic potential of submucosal invasive colorectal carcinoma. Oncology. 1995 March-April; 52(2): 134-9.
9. Aoki R, Tanaka S, Haruma K, Yoshihara M, Sumii K, Kajiyama G, Shimamoto F, Kohno N. MUC-1 expression as a predictor of the curative endoscopic treatment of submucosally invasive colorectal carcinoma. Dis Colon Rectum. 1998 October; 41(10):1262-72.
10. Yamamoto H, Iku S, Adachi Y, Imsumran A, Taniguchi H, Nosho K, Horiuchi S, Yoshida M, Itoh F, Imai K. Association of trypsin expression with tumour progression and matrilysin expression in human colorectal cancer. J. Pathol. 2003 February; 199(2):176-84.
11. Frederiksen C M, Knudsen S, Laurberg S, Orntoft T F. Classification of Dukes B and C colorectal Cancers using expression arrays. J Cancer Res Clin Oncol (2003) 129:263-271.
12. Kwon H C, Kim S H, Roh M S, Kim J S, Lee H S, Choi H J, Jeong J S, Kim H J, Hwang T H. Gene expression profiling in lymph node positive and lymph node negative colorectal cancer. Dis Colon Rectum (2004) 47:141-152.
13. Friederichs J, Rosenberg R, Mages J, Janssen K P, Maeckl C, Nekarda H, Holzmann B, Siewert J R. Gene expression profiles of different clinical stages of colorectal carcinoma: toward a molecular genetic understanding of tumor progression. Int J Colorectal Dis (2005) 20:391-402.
14. Koehler A, Bataille F, Schmid C, Ruemmele P, Waldeck A, Blaszyk H, Hartmann A, Hofstaedter F, Dietmaier W. Gene expression profiling of colorectal cancer and metastases divides tumours according to their clinopathological stage. J Pathol (2004) 204:65-74.
15. Croner R S, Foertsch T, Brückl W M, Siebenhaar R, Günther K, Hlubek F, Stremmel C, Hohenberger W, Reingruber B. Tissue preparation for gene expression profiling of colorectal carcinoma—three alternatives to laser microdissection with preamplification. J Lab Clin Med 2004 June; 143 (6):344-351.
16. Croner R S, Peters A, Brueckl W M, Matzel K E, Klein-Hitpass L, Brabletz T, Papadopoulos T, Hohenberger W, Reingruber B, Lausen B. Microarray versus conventional prediction of lymph node metastasis in colorectal carcinoma. Cancer 2005; 104(2); 395-404.

1. A micro-array for the prediction of a lymph node metastasis in connection with colorectal carcinoma, wherein the micro-array comprises one or more probes that specifically hybridize to one or more of SEQ ID NOs: 1, 2, 19, 22, 36, 42, 57, 58, 61, 80, 81, 85, 132, 152, 162, 168, 175, 184-186, 200, 206-216, and 223-240, and complements, reverse, and reverse complements thereof.

2. The micro-array according to claim 1, wherein the micro-array further comprises one or more additional probes that specifically hybridize to one or more of SEQ ID NOs: 1-240.

3. The micro-array according to claim 1, wherein the one or more probes present on the micro-array are chemically modified.

4. The micro-array according to claim 3, wherein the one or more probes present on the micro-array are marked.

5. The micro-array according to claim 4, wherein the one or more probes each comprise a marker that is selected from the group consisting of a radioactive marker, a fluorescence marker, a biotin marker, a digoxigenin marker, a peroxidase marker, and a marker that comprises an alkaline phosphatase.

6. The micro-array according to claim 1, wherein the one or more probes of the micro-array are bound to a solid phase matrix.

7. The micro-array according to claim 6, wherein the micro-array is a DNA, RNA, or PNA array.

8. An ex vivo method for predicting a lymph node metastasis in connection with colorectal carcinoma, the method comprising:
providing a patient with a colorectal carcinoma comprising tumor tissue;
isolating a sample of the tumor tissue of the colorectal carcinoma from the patient;
extracting the RNA from the sample;
optionally transcribing the RNA into cDNA or cRNA;
determining if the sample contains an RNA which corresponds to a nucleotide sequence selected from the group consisting of SEQ ID NOs: 1-240,
wherein the determining step is predictive of a lymph node metastasis in the patient.

9. The method according to claim 8, wherein the determining step is carried out by RT-PCR.

10. The method according to claim 8, wherein the determining step is carried out by employing complementary nucleic acid probes.

11. The method according to claim 10, wherein the determining step comprises detecting nucleic acids with nucleic acid probes that specifically hybridize to a segment of each of the nucleic acids; their complementary, reverse, or reverse-complementary sequences; to RNA sequences derived therefrom; or to derivatives thereof.

12. The method according to claim 8, wherein the determining comprises contacting the RNA, or the cDNA or cRNA transcribed therefrom, with a hybridizing nucleotide sequence under moderately stringent conditions.

13. The method according to claim 8, wherein the determining step comprises detecting the presence of a protein encoded by an RNA present in the sample.

14. A method for detecting expression in a tumor in a subject of a nucleic acid comprising a nucleotide sequence as set forth in one or more of SEQ ID NOs: 1-240, the method comprising:
a) extracting RNA from a sample of tumor tissue isolated from a subject;
b) applying the RNA, or a cRNA or a cDNA produced therefrom, to a micro-array, wherein the micro-array comprises one or more probes that specifically hybridize to one or more of SEQ ID NOs: 1, 2, 19, 22, 36, 42, 57, 58, 61, 80, 81, 85, 132, 152, 162, 168, 175, 184-186, 200, 206-216, and 223-240; and
c) detecting hybridization of the RNA, or of the cRNA or the cDNA produced therefrom to one or more of the one or more probes present on the micro-array.

15. A protein micro-array for the prediction of a lymph node metastasis in connection with colorectal carcinoma, wherein the micro-array is able to detect one or more amino acid sequences encoded by one or more of SEQ ID NOs: 1-240.

16. The protein micro-array according to claim 15, wherein the micro-array is an antibody micro-array or a Western Blot micro-array.

17. An inhibitor or modulator of one or more of SEQ ID NOs: 1-240, or of a protein encoded thereby.

18. A pharmaceutical composition comprising one or more inhibitors or modulators according to claim 17 and a pharmaceutically acceptable carrier.

19. The micro-array according to claim 6, wherein the solid phase matrix is selected from the group consisting of a nylon membrane, glass, and a plastic material.

20. The method according to claim 10, wherein the complementary nucleic acid probes comprise a cDNA or an oligonucleotide probe.
